(19) United States
(12) Patent Application Publication
(10) Pub. No.: US 2017/0165485 A1
(43) Pub. Date: Jun. 15, 2017

(54) SYSTEMS AND METHODS FOR NON-INVASIVE TREATMENT OF HEAD PAIN

(71) Applicants: Michael J. Sullivan, Richfield, MN (US); Bryan Allen Clark, Forest Lake, MN (US); John Huss, Zimmerman, MN (US); Benjamin Y. Arcand, Minneapolis, MN (US); Jai Shetake, Santa Clarita, CA (US); Bryce Calvin Beverlin, II, St. Paul, MN (US)

(72) Inventors: Michael J. Sullivan, Richfield, MN (US); Bryan Allen Clark, Forest Lake, MN (US); John Huss, Zimmerman, MN (US); Benjamin Y. Arcand, Minneapolis, MN (US); Jai Shetake, Santa Clarita, CA (US); Bryce Calvin Beverlin, II, St. Paul, MN (US)

(21) Appl. No.: 15/371,177

(22) Filed: Dec. 6, 2016

Related U.S. Application Data
(60) Provisional application No. 62/267,435, filed on Dec. 15, 2015.

(51) Int. Cl. A61N 1/36; A61B 5/00; A61N 7/00; A61N 1/28; A61N 2/00; A61N 1/20

(52) U.S. Cl. CPC A61N 1/36021; A61N 2/006; A61N 1/20; A61N 1/36025; A61N 2/008; A61N 7/00; A61N 1/28; A61B 5/0022; A61N 2001/34

(57) ABSTRACT

Systems and methods for non-invasive management of head pain are disclosed. The system includes a headgear configured to be worn on a patient's head. The headgear can include a base and an extension coupled to the base, and a number of therapy devices removably or adjustably attached to the base or the extension. The therapy devices can deliver various modes of therapeutic energy at respective target sites on the head, including neuromodulation of peripheral pain pathways and/or the cerebral cortex, and therapy modalities to facilitate or enhance the neuromodulation effects. The system can include a portable device that enables the user to control the therapy devices on the headgear. The user can use the portable device to optionally access a web-based repository to acquire information about headgear usage from other users, and use that information to guide the programming of the therapy devices.

### CLAIM OF PRIORITY

This application claims the benefit of priority under 35 U.S.C. §119(e) of U.S. Provisional Patent Application Ser. No. 62/267,435, filed on Dec. 15, 2015, which is herein incorporated by reference in its entirety.

### TECHNICAL FIELD

This document relates generally to medical devices, and more particularly, to systems, devices, and methods for delivering therapies to treat head pain.

### BACKGROUND

Head pain may be experienced only in a part of the head or over the entire head. Clinically, head pain may include migraine, cluster headache, tension headache, or sinus headache, among other types of headaches. Migraine is among the most common patient complaints encountered in neurology practice and is a leading cause of emergency room visits. Migraine symptoms may include a pounding headache, nausea, vomiting, and light sensitivity. Approximately 20% of migraine patients may experience an aura, most commonly a visual aura. Migraine can be episodic (e.g., less than 15 days per month), or it can be chronic (e.g., 15 or more days per month).

Head pain may be initiated via inflammation in the dural-vascular structures of the cortex, such as meninges and surrounding vessels. The pain signals may originate from the front, side, or rear of the head, and transmit to the central nervous system (CNS) by cranial or spinal peripheral pathways. In particular, the head pain signals may pass through a trigeminal cervical complex (TCC) which is known as the “gateway” or “relay center” of nociceptive head pain, and ascend through the brainstem and thalamus before ultimately being received and cognitively processed by the sensory cortex of the brain.

Based on a theory known as the “gate control” mechanism, a pain signal that dominates the peripheral pathway may pass through a pain gate located at the junction of the peripheral and central nervous systems, such as the dorsal horn of the spinal cord. In the case of headache pain, the TCC region acts as a pain gate. If activated peripheral-nerve pain-fiber signals dominate the pathway, then the pain gate may open such that the pain signals can pass through and reach the CNS. Conversely, if the activated pain fibers are swamped by non-noxious peripheral stimuli, then the pain gate may be closed and the non-noxious signals can be sent to the CNS to be evaluated, at the expense of the pain signals.

Head pain may also be related to a process of “neural sensitization” by which neurons become increasingly responsive to nociceptive and non-nociceptive stimulation as a result of exposure to repeated noxious peripheral stimulation (e.g., excess heat or pressure). Sensitization results in decreased response (or activation) thresholds, increased response magnitude, and expansion of receptive fields. Central sensitization can lead to the development of spontaneous neuronal activity even in the absence of a noxious peripheral stimulus, and/or a sustained response to a peripheral noxious stimulus even after the peripheral stimulus is removed. Such a characteristic of plasticity of the CNS may lead to hypersensitivity to pain by changing the sensory response elicited by normal inputs that would usually evoke innocuous sensations.

Head pain such as migraine has conventionally been treated with medications including pain relievers and anti-nausea drugs. Neuromodulation has been proposed as a therapy for head pain. Implantable neuromodulators with leads implanted in the tissue over the occipital nerves for delivering invasive occipital nerve stimulation (ONS) have shown promise as a treatment for headaches such as migraine, cluster headaches, or cervicogenic headaches. Various other minimally-invasive and non-invasive neuromodulation therapies have also been evaluated.

### SUMMARY

In Example 1, a system for non-invasive management of head pain is disclosed. The system can comprise a headgear that can be worn on a patient's head. The headgear can include a base, an extension coupled to the base via one or more connectors, at least first and second therapy devices removably or adjustably attached to one or more of the base or the extension, and a first controller attached to the base. The first therapy device can deliver a first mode of therapeutic energy to a first target site on the head, and the second therapy device can deliver a second mode of therapeutic energy to a second target site on the head. The first controller can control at least the first and second therapy devices for delivering the respective first and second modes of therapeutic energy to the respective first and second target sites.

Example 2 can include, or can optionally be combined with the subject matter of Example 1 to optionally include, the extension that can include one or more frame elements detachably or adjustably connected to the base via the one or more connectors. One or more of the at least first and second therapy devices can be removably or adjustably attached to the one or more frame elements.

Example 3 can include, or can optionally be combined with the subject matter of Example 2 to optionally include, the first therapy device that can be removably or adjustably attached to the base curved to conform to at least a portion of a transverse circumference of the head, and the second therapy device that can be removably or adjustably attached to one of the one or more frame elements of the extension, the one frame element curved to conform to at least a portion of a coronal circumference or a portion of a sagittal circumference of the head.

Example 4 can include, or can optionally be combined with the subject matter of one or any combination of Examples 1 through 3 to include, the extension that can include a cover securely interconnecting the base and the one or more frame elements of the extension.

Example 5 can include, or can optionally be combined with the subject matter of Example 2 to optionally include, the base that can include an eyepiece positioned close to one or both eyes of the patient, at least two side elements connected to opposite sides of the eyepiece, and a rear element connected to the two sidebars. The one or more frame elements can include at least a cross element removably or adjustably attached to the two sidebars of the first frame element.

Example 6 can include, or can optionally be combined with the subject matter of one or any combination of Examples 1 through 5 to include, the first and second therapy devices that can be respectively selected from the group consisting of: a first neuromodulator configured to deliver transcutaneous electrical nerve stimulation (TENS); a second neuromodulator configured to deliver transcranial direct current stimulation (tDCS); a third neuromodulator configured to deliver transcranial magnetic stimulation (TMS); a thermal energy transducer configured to generate thermal energy; an ultrasound transducer configured to generate ultrasonic energy; a vibrotactile transducer configured to generate vibration or tactile massage; a radio-frequency (RF) transducer configured to generate high-frequency RF energy; a laser device configured to generate laser light energy; a visual stimulator configured to generate or prevent visual stimulation; and an aural stimulator configured to generate aural stimulation.

Example 7 can include, or can optionally be combined with the subject matter of Example 6 to optionally include, the first therapy device that can include the first neuromodulator configured to deliver TENS to a first target site innervated by a trigeminal nerve or an occipital nerve, and the second therapy device that can include the second neuromodulator configured to deliver tDCS to a second target site corresponding to a motor cortex target of the head.

Example 8 can include, or can optionally be combined with the subject matter of one or any combination of Examples 6 or 7 to include, the TENS that can include a pulse train with one or more time-variant stimulation parameters.

Example 9 can include, or can optionally be combined with the subject matter of one or any combination of Examples 1 through 8 to include, electrodes coupled to the at least first and second therapy devices and configured to be in close contact with scalp or skin at the first and second site. The first and second therapy devices can deliver the respective first or second mode of therapeutic energy via the electrodes.

Example 10 can include, or can optionally be combined with the subject matter of one or any combination of Examples 1 through 9 to include, a portable device that can include a communicator to communicate with the at least first and second therapy devices on the headgear via a transmitter-receiver circuit of the headgear, a user interface including a display and an input device configured to receive user input, a second controller coupled to the user interface and the communicator, and a memory to store personal therapy information of the patient including the one or more therapy parameters. The second controller can include a programmer circuit that can generate one or more therapy parameters associated with the delivery of the first and second modes of therapeutic energy.

Example 11 can include, or can optionally be combined with the subject matter of Example 10 to optionally include, the portable device that can be a personal mobile communication device.

Example 12 can include, or can optionally be combined with the subject matter of one or any combination of Examples 10 or 11 to include, the portable device that can be communicatively coupled to the headgear through a wired or wireless connection.

Example 13 can include, or can optionally be combined with the subject matter of one or any combination of Examples 10 through 12 to include, one or more physiological sensors coupled to the portable device and configured to sense respective one or more physiological signals. The portable device can include a signal processor to detect from the one or more physiological signals a physiological event. The programmer circuit of the portable device can initiate, or adjust the parameters associated with, the delivery of the respective first and second modes of therapeutic energy in response to the detected physiological event.

Example 14 can include, or can optionally be combined with the subject matter of Example 13 to optionally include, the one or more physiological sensors that can sense one or more of a heart rate signal, a pulse rate signal, a hemodynamic signal, a heart rate variability signal, a galvanic skin response signal, an electromyogram signal, a saliva production signal, or an electroencephalogram signal.

Example 15 can include, or can optionally be combined with the subject matter of one or any combination of Examples 10 through 14 to include, the portal device that can be communicatively connected to a web-based data repository for storing public therapy information obtained from two or more users. The portable can enable the patient to access content in the web-based data repository, upload personal therapy information of the patient onto the data repository, or download from the data repository the public therapy information into the portable device for storing in the memory or to use in programming the at least first and second therapy devices.

In Example 16, a method for non-invasively managing head pain in a patient using a headgear wearable on a head of a patient is disclosed. The method can include steps of placing the headgear on the patient's head, positioning at least a first therapy device removably or adjustably attached the headgear to a first target site on the head, positioning at least a second therapy device removably or adjustably attached the headgear to a second site on the head, delivering a first mode of therapeutic energy at the first target site using the at least first therapy device, and delivering a second mode of therapeutic energy at the second target site using the at least second therapy device.

Example 17 can include, or can optionally be combined with the subject matter of Example 16 to optionally include, steps of providing a portable device configured to communicate with the at least first and second therapy devices on the headgear, establishing a communication between the at least first and second therapy device and the portable device, and programming the at least first and second therapy device using the portable device. The first and second modes of therapeutic energy can be delivered using the respective first and second therapy devices according to the programming.

Example 18 can include, or can optionally be combined with the subject matter of Example 17 to optionally include, establishing the communication between the at least first and second therapy device and the portable device via a wired or wireless connection.

Example 19 can include, or can optionally be combined with the subject matter of Example 16 to optionally include, delivering the first and second modes of therapeutic energy respectively selected from the group consisting of: a transcutaneous electrical nerve stimulation (TENS); a transcranial direct current stimulation (tDCS); a transcranial magnetic stimulation (TMS); a thermal energy mode; an ultrasonic energy mode; a vibration or tactile massage mode; a high-frequency RF energy mode; a laser light energy mode; a visual stimulation; and an aural stimulation.

Example 20 can include, or can optionally be combined with the subject matter of Example 19 to optionally include, delivering the first mode of therapeutic energy including the TENS to a first target site innervated by a trigeminal nerve or an occipital nerve, and delivering the second mode of therapeutic energy includes the tDCS to a second target site corresponding to a motor cortex target of the head.

Example 21 can include, or can optionally be combined with the subject matter of Example 20 to optionally include, delivering the TENS including a pulse train with one or more time-variant stimulation parameters.

Example 22 can include, or can optionally be combined with the subject matter of Example 16 to optionally include, detecting a physiological event. The delivery of the first and second modes of therapeutic energy includes initiating, or adjusting one or more parameters associated with, the delivery of the respective first and second modes of therapeutic energy in response to the detection of the physiological event.

Example 23 can include, or can optionally be combined with the subject matter of Example 16 to optionally include, one or more steps of establishing a communication between the portable device and a web-based data repository that stores public therapy information obtained from two or more users, using the portable device to access content in the web-based data repository, uploading personal therapy information of the patient onto the data repository, or downloading from the data repository the public therapy information into the portable device for storing in the portable device or programming the at least first and second therapy devices.

This Summary is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof, each of which are not to be taken in a limiting sense. The scope of the present invention is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, a schematic drawing of a headgear configured to be worn on a head of a patient.

FIG. 2 illustrates, by way of example and not limitation, an example of a therapeutic cap for use in a system for non-invasive management of head pain.

FIG. 3 illustrates, by way of example and not limitation, an example of a therapeutic eyewear for use in a system for non-invasive management of head pain.

FIGS. 4A-B illustrate, by way of example and not limitation, an example of a therapeutic headgear and a part of the environment in which it can be used.

FIGS. 5A-B illustrate, by way of example and not limitation, an example of a type of electrode assembly for delivering neuromodulation therapy for alleviating head pain.

FIGS. 6A-B illustrate, by way of example and not limitation, an example of another type of electrode assembly for delivering neuromodulation therapy for alleviating head pain.

FIG. 7 illustrates, by way of example and not limitation, an example of a system for non-invasive management of head pain.

FIG. 8 illustrates, by way of example and not limitation, another example of a system for non-invasive management of head pain.

FIG. 9 illustrates, by way of example and not limitation, an example of a mobile communication device for controlling a headgear for head pain management.

FIG. 10 illustrates, by way of example and not limitation, an example of a method for non-invasive management of head pain using a headgear wearable on a head of a patient.

FIG. 11 illustrates, by way of example and not limitation, an example of a method for non-invasive management of head pain using a system comprising a headgear and a portable device.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

There is a need to improve non-invasive head pain management, such as a reduction of frequency, intensity, or duration of migraine events. For example, pharmaceutical and device-based therapies for head pain, although effective in some patients, still pose certain disadvantages. Pain medications may have undesirable side effects. Some patients may not obtain satisfactory relief of head pain and associated discomfort. Implantable neuromodulation therapy such as DBS typically requires penetration of a patient's skull and surgical implantation of a lead with electrodes into the brain. External non-invasive neurostimulation therapies, such as a therapeutic headband or wearable devices, typically target only a particular mechanism implicated in head pain. However, it is now understood that migraine or other primary head pain may involve numerous mechanisms and may be very specific to the individual. A therapy modality that targets one particular pain mechanism may not be desirable for variety of patients with head pain of different mechanisms. For example, a cranial neurostimulator with supra-orbital electrodes acts on a trigeminal nerve but does not have an effect on the occipital nerve, and thus may not replicate the implantable occipital neurostimulator for treating certain types of head pain such as occipital neuralgia. Furthermore, hair may reduce the reliability of electrode-tissue contact and impact the therapy efficacy.

Available external neurostimulation devices also lack flexibility. For example, such devices are typically limited to a certain type of therapy (e.g., electrostimulation) with limited configuration options (e.g., limited number of target sites and therapy parameters used for electrostimulation). Other than some drugs for some patients, typically no treatment from such devices would be applied after a patient detects an aura prior to a headache to prevent the occurrence of pain and other undesirable manifestations of that migraine headache. The pain relief treatment may not be an effective preventive pain therapy for avoiding onset of the migraine headache. Other potential disadvantages may include lack of adequate and intuitive user control of the device such as adjustment of the therapy parameters, rigidity of the device (e.g., a head-wearing neuromodulator) which prevents it from comfortably fitting different head sizes, and device reliability and connection integrity while the device is being used.

Embodiments of the present subject matter provide systems, devices, and methods to non-invasively treat and prevent head pain. The system and devices can provide various therapy modalities including electrostimulation therapy modulating the peripheral pain pathways and/or the cerebral cortex, as well as other therapy modalities to facilitate or enhance the neuromodulation effects. The system can include a handheld device that enables the user to control the therapy delivery and/or to access a web-based repository to acquire information for guiding the programming of the devices for managing head pain.

Disclosed herein are systems and methods for non-invasive management of head pain. The system includes a headgear configured to be worn on the patient's head. The headgear can include a base and an extension coupled to the base, and a number of therapy devices removably or adjustably attached to the base or the extension. The therapy devices can deliver various modes of therapeutic energy at respective target sites on the head, including neuromodulation of peripheral pain pathways and/or the cerebral cortex and therapy modalities to facilitate or enhance the neuromodulation effects. The system can include a portable device that enables the user to control the therapy devices on the headgear. The user can use the portable device to optionally access a web-based repository to acquire information about headgear usage from other users, and use that information to guide the programming of the therapy devices.

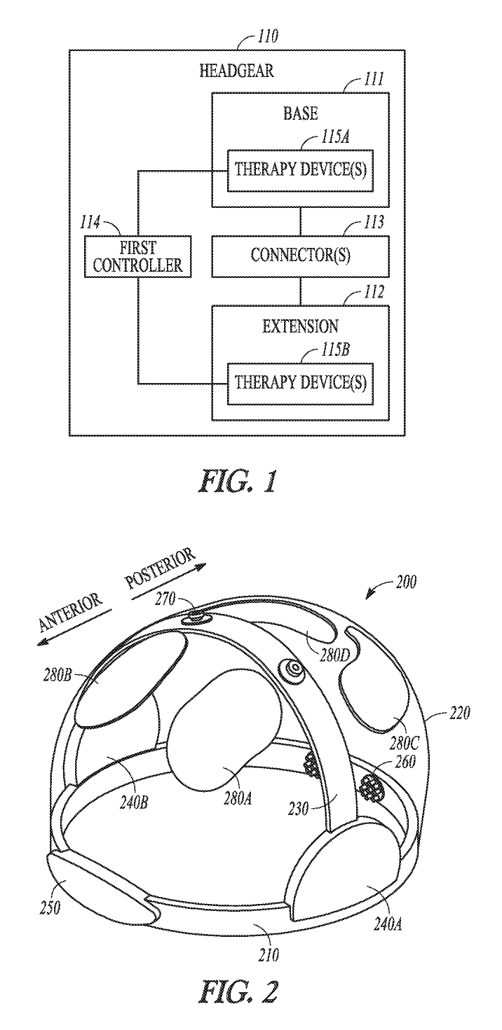

FIG. 1 illustrates, by way of example and not limitation, a schematic drawing of a headgear 110 configured to be worn on a patient's head. The headgear 110 can be a part of a non-invasive head pain management system for treating or preventing head pain such as migraine, cluster headache, tension headache, or sinus headache, among other types of headaches. The headgear 110 may include a base 111, an extension 112 coupled to the base 111 via one or more connectors 113, and a first controller 114 which may be attached to the base 111 or the extension 112. The base 111 may be sized and shaped to fit at least a portion of the head of the patient. The base 111 may be made of elastic material, such as to stretchably conform to at least a portion of a circumference of head. Examples of the elastic material may include woven fabric, extensible non-woven fabric, or polymer, among other materials. In an example, the base 111 may have a pre-defined shape of a ring or a portion of a ring. The base 111 may include at least an elastic portion along the base 111, such as an elastic strap interconnecting two points on the base 111. In an example, the base 111 may include an adjustable coupling mechanism, such as one or more hooks, buttons, buckles, clips, or other types of fasteners. The elastic portion or the adjustable coupling mechanism may allow the base 111 to securely fit to different head sizes.

The extension 112 may include one or more frame elements curved to fit at least a portion of the head, such as a portion of a coronal circumference or a sagittal circumference of the head. In an example, like the base 111, one or more of the frame elements may be made of elastic material to stretchably conform to at least a portion of a circumference of head. In an example, the frame elements can have of the shape of a band or bar. One or more of the frame elements of the extension 112 can be detachably or adjustably connected to the base 111 via the one or more connectors 113. Examples of the connectors 113 can include a snap-fit coupling, a threaded or other rotation or screw-in coupling, a slide-in engagement, a hinge, or other adjustable coupling mechanisms. In an example, a plurality of coupling interfaces are made available on the base 111, which enable two or more frame elements to be individually engaged to, or disengaged from, the base 111. The connectors may provide a mechanical connection and an electrical connection. The electrical connection may include a power connection and/or a communication connection. An example of an electrical connection is a universal serial bus (USB) connection. By way of example and not limitation, various embodiments use a micro USB connector to provide an electrical connection.

As illustrated in FIG. 1, two or more therapy devices 115A-B may be removably or adjustably attached to one or more of the base or the extension. Each therapy device can be configured to deliver a specified mode of therapeutic energy at a specified target site on the head to treat, alleviate, or prevent head pain. Examples of the therapy devices 115A-B can include neuromodulators such as stimulators configured for providing electrostimulation or magnetic stimulation, or transducers that can transform one type of energy (e.g., electrical energy) to another different type of energy, such as thermal energy, radio-frequency energy, ultrasonic energy, vibrotactile energy, or laser light energy, among others. The therapy devices 115A-B can be moved about the base or the extension, either when the headgear 110 is not being worn or when the headgear 110 is worn on the head, and repositioned at desired locations. In an example, the therapy devices 115B on the extension 112 can be removably or adjustably attached to the one or more frame elements. The adjustable positions of the therapy devices 115A-B with respect to the extension 112 or the base 111, and the frame elements that are detachable and adjustable with respect to the base 111, may provide enhanced flexibility in applying head pain therapies with desired number and location of the therapy sites.

The first controller 114 may be configured to control the two or more therapy devices 115A-B for delivering the respective modes of therapeutic energy to the respective target sites. Examples of the headgear, the therapy devices, and therapeutic energy modes are discussed below, such as with reference to FIGS. 2-4.

FIG. 2 illustrates, by way of example and not limitation, an example of a therapeutic cap 200 that may be used in a system for non-invasive management of head pain. The therapeutic cap 200 is an embodiment of the headgear 110 in FIG. 1, and may include a base 210, an extension including a cover 220 and at least one frame element 230, controller compartments 240A-B, and a plurality of therapy devices 250, 260, 270 and 280A-D.

The base 210 may have a ring-shaped frame that is sized to fit the transverse circumference of the head of the patient. The base 210 may have an ergonomic design and may include an adjustment mechanism to allow the base 210 to adjustably and securely fit to different head sizes. In an example, as discussed with reference to the headgear 110 in FIG. 1, at least a portion of the base 210 can be made of elastic material to stretchably conform to different head sizes.

The cover 220 may be made of fabric, polymer, or other flexible material to securely interconnect the base 210 and the frame element 230. The frame element 230, which is an embodiment of the frame element of the headgear 110, may be detachably and adjustably connected to the base 210 via one or more connectors such as located inside the controller compartments 240A-B. In an example, the connectors may include a swivel or other rotatory couplings between the base 210 and the frame element 230 to enable the frame element 230 to rotate, with respect to the base 210, toward the anterior or posterior side of the head.

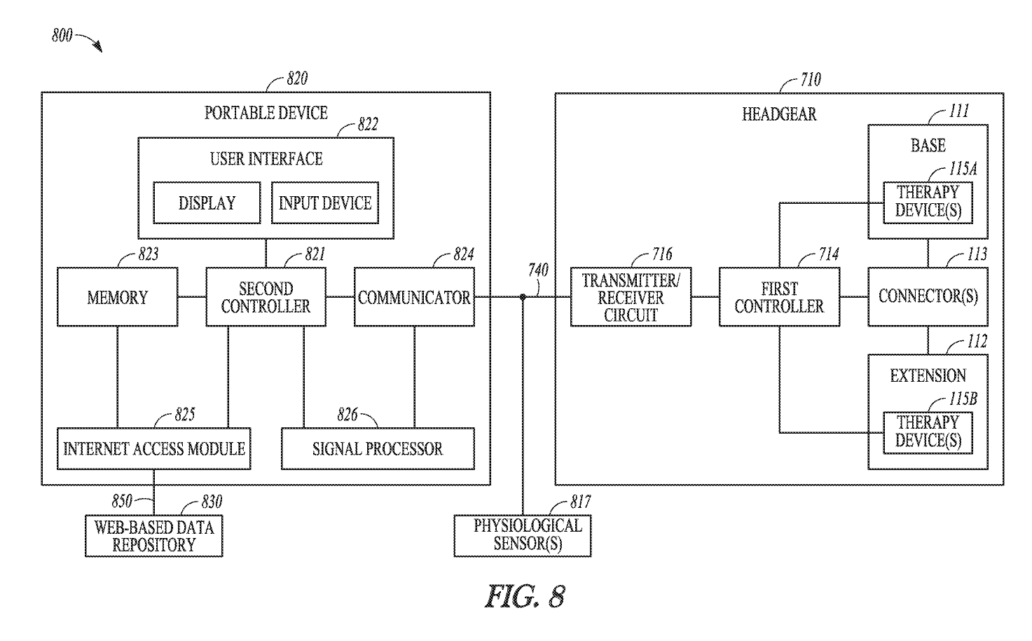

The control compartments 240A-B can be located on one or both sides of the therapeutic cap 200, such as where the base 210 and the frame element 230 interconnect with each other. In an example, only one control compartment, such as 240A or 240B, may be provided for the therapeutic cap 200. In addition to the connectors that connect the base 210 and the frame element 230, one or both of the control compartments 240A-B can include electrical and mechanical coupling interfaces with the therapy devices 250, 260, 270, and 280A-D, and control circuitry that controls the therapy devices for delivering respective therapeutic energy at the respective target sites. One or both of the control compartments 240A-B can include one or more power supplies, such as one or more batteries or rechargeable batteries that respectively provide power to the therapy devices 250, 260, 270, and 280A-D. The one or both of the control compartments 240A-B can include transducers that provide therapeutic energies such as acoustic signals including auditory sounds for patient relaxation to facilitate pain relief. In some examples, one or both of the control compartments 240A-B can include an interface and a communication module for connecting and communicating with an external device, or for charging the one or more rechargeable batteries in the therapeutic cap 200. In some examples, the one or both of the control compartments 240A-B may include physiological sensors for sensing physiological signals from the head. Examples of the physiological sensors for use in the headgear are discussed below, such as with reference to FIG. 8. In an example, a set of switches can be included in one or both of the control compartments 240A-B which enable the user to override some or all therapies, shut down the communication, or power off the therapeutic cap 200 for patient safety during emergencies.

The therapy devices 250 and 260 may each include one or more neuromodulators configured to generate and deliver transcutaneous electrical nerve stimulation (TENS). By way of non-limiting examples, and as illustrated in FIG. 2, the therapy device 250 may be attached to anterior portion of the base 210, and the therapy device 260 may be attached to the posterior portion of the base 210. The base 210 may be sized and shaped such that the anterior portion of the base 210 is positioned at or next to the supra-orbital region of the forehead of the patient, and the posterior portion of the base 210 is positioned at the occipital region of the head. The therapy device 250 can deliver TENS to a supra-orbital site innervated by one or both trigeminal nerves, and the therapy device 260 can deliver TENS to an occipital site innervated by an occipital nerve. Both the therapy devices 250 and 260 can be coupled to respective one or more electrodes positioned at, and in close contact with, scalp or skin at the respective target sites.

TENS may disrupt the mechanism causing head pain (such as migraine episodes) by swamping the pain fibers with non-noxious peripheral stimuli, thereby shutting the gate at the junction of the peripheral and central nervous system, and blocking the pathway (such as via the trigeminal nerve and/or the occipital nerve) leading to the CNS where the pain sensation is interpreted. In an example, the TENS may be delivered using a pulse train with one or more time-variant stimulation parameters. Invariant stimulation, such as using constant amplitude, frequency, or other stimulation parameters, may cause complications due to cellular and synaptic mechanisms. Such complications may include neural accommodation, adaptation, habituation, or stimulus generalization, which may cause diminished neural response to stimulation and degraded therapeutic efficacy over time. Electrostimulation with one or more time-variant stimulation parameters may reduce the likelihood of accommodation and habituation. The time-variant stimulation parameters can include time-variant duty cycle, time-variant pulse amplitude, time-variant pulse width, time-variant frequency, or time-variant stimulation duration, among others. In an example, the time-variant stimulation parameters can include a random stimulation parameter, such as random burst duty cycles.

The therapy device 270 may include one or more neuromodulators configured to deliver transcranial direct current stimulation (tDCS). By way of non-limiting examples, and as illustrated in FIG. 2, the therapy device 270 can be attached to the frame element 230. The frame element 230 can have a shape of a flat panel or band curved to conform to at least a portion of the coronal circumference. The therapy device 270 can move around the frame element 230 and be securely positioned at a target site. Similar to the therapy devices 250 and 260, the therapy device 270 can be coupled to one or more electrodes positioned at, and in close contact with, scalp at the target sites corresponding to a motor cortex target of the head, and externally deliver the tDCS to the motor cortex region.

The central sensation of pain at the CNS may be caused by the plasticity of the CNS, which may include increased spontaneous neuronal activity and sustained response to peripheral noxious stimuli. The tDCS via motor cortex may impact indirectly, through interaction with interneurons, deep-brain structures associated with head-pain transmittal, thereby reversing central sensitization of pain. In an example, TENS such as provided by the therapy devices 250 and 260 and tDCS such as provided by the therapy device 270 may be delivered in an alternating mode. For example, TENS can be delivered for a first duration (e.g., 10 seconds) followed by the tDCS for a second duration (e.g., 10 second).

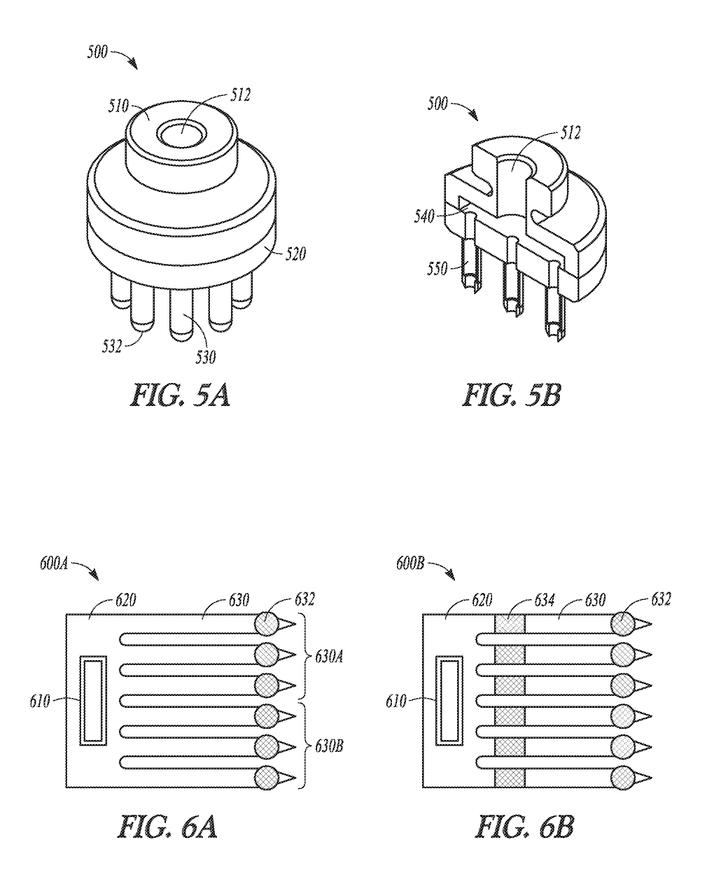

The electrodes coupled to the therapy devices 250, 260, or 270 can be configured to have reliable conductivity at the electrode-tissue interface, such as the target scalp sites covered by hair. In an example, an anode electrode and a cathode electrode can be provided and coupled the device 270 to provide bipolar electrostimulation of the motor cortex. By way of non-limiting examples, the anode electrode may be placed over the motor cortex, contralateral to the most painful side of the head (or the side where the symptoms begin), and the cathode electrode may be placed over the supraorbital area pertaining to the most painful side of the head (or side where the symptoms begin), such as corresponding to the frontal-parietal lobe on the left or the right cerebral hemisphere. In some examples, the electrode positions can also be determined according to the dominant pain location. Examples of the electrodes for delivering electrostimulation are discussed below, such as with reference to FIGS. 5-6.

The therapeutic cap 200 can include the therapy devices 280A-D that may provide various modalities of therapy to induce a state of relaxation or relief in the patient such as to achieve therapeutic effect of pain relief. Examples of the therapy devices 280A-D can include a third neuromodulator configured to deliver transcranial magnetic stimulation (TMS), a thermal energy transducer configured to generate thermal energy, an ultrasound transducer configured to generate ultrasonic energy, a vibrotactile transducer configured to generate vibration or tactile massage, a radio-frequency (RF) transducer configured to generate high-frequency RF energy, a laser device configured to generate laser light energy, a visual stimulator configured to generate or prevent visual stimulation, or an aural stimulator configured to generate aural stimulation, among others. Such therapies as provided by the therapy devices 280A-D can be used in addition to or in lieu of the electrostimulation, such as TENS or tDCS provided by the therapy devices 250, 260, or 270. By way of non-limiting examples, and as illustrated in FIG. 2, the therapy devices 280A-D may be attached to the cover 220 and adjustably positioned to one or more target scalp sites on the frontal, temporal, or parietal side of the head. In an example, at least one of the therapy devices 280A-D may be positioned on one or more of the base 210 or the frame element 230, or inside the control compartments 240A-B. In some examples, one or more of the therapy devices 280A-D can be distributed between the cover 220 and the control compartments 240A-B. For example, one of the therapy devices 280A-D may have an energy-conversion module residing within the control compartments 240A-B. In an example, one or more therapeutic or sensory element (such as a thermal pad for thermal therapy, or a vibrational element for vibration or tactile therapy) may be positioned on the cover 220 to provide contact with scalp or skin. The control compartments 240A-B can include one or more power supplies that respectively provide power to the therapy devices 280A-D. In an example, the power supplies may include one or more rechargeable batteries.

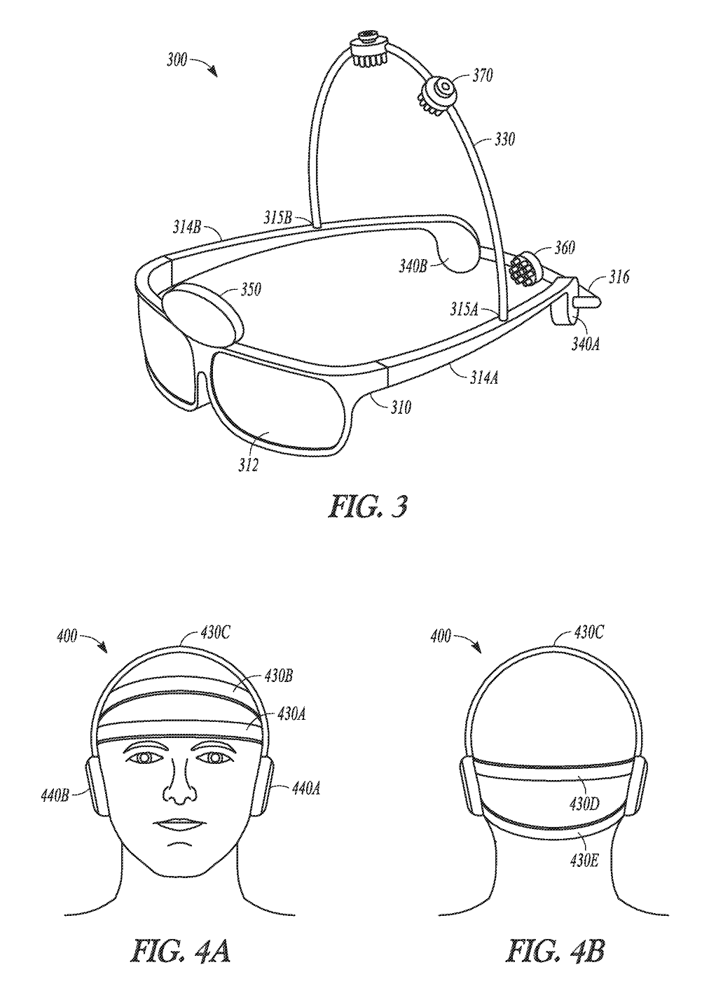

FIG. 3 illustrates, by way of example and not limitation, an example of a therapeutic eyewear 300 that may be used in a system for non-invasive management of head pain. The therapeutic eyewear 300 is an embodiment of the headgear 110 in FIG. 1, and may include a base 310, an extension including at least one cross element 330, two controller compartments 340A-B, and a plurality of therapy devices 350, 360, and 370.

The base 310 has a shape analogous to eyeglasses, and may include an eyepiece 312 positioned close to one or both eyes of the patient, at least two side elements 314A-B connected to opposite sides of the eyepiece 312, and a rear element 316 connected to the two side elements 314A-B. The eyepiece 312, the at least two side elements 314A-B, and the rear element 316 are interconnected to form a loop adjustably conforming to at least a portion of a transverse circumference of the head.

The eyepiece 312 may include tinted shades to shield patient eyes from light such as to provide relaxation and facilitate therapeutic relief from electrostimulation. The tinted shades can be at least partially enclosed by respective rims. In an example, the eyepiece 312 may include a transducer, such as positioned at the rims, which are configured to generate or prevent visual stimuli to induce a state of relaxation or relief in the patient. A nosepiece, analogous to nose pads in eyeglasses, can be provided to index device for appropriate electrode placement. A therapy device 350 can be adjustably attached to the eyepiece 312, such as at a connective bridge between the rims, to provide therapy to alleviate head pain. In an example, the therapy device 350 can be positioned at or next to the supra-orbital region of the patient's forehead, and to deliver TENS to a supra-orbital site innervated by one or both trigeminal nerves.

The side elements 314A-B can be coupled to the eyepiece 312 via respective connectors such as hinges. The side elements 314A-B can include one or more attachment points to allow one or more therapy devices to be detachably attached to the side elements 314A-B. By way of non-limiting examples, and as illustrated in FIG. 3, cross element 330 can be removably or adjustably attached to the side elements 314A-B via connectors at respective attachment positions 315A-B on the side elements 314A-B. The cross element 330, which is an embodiment of the frame element of the headgear 110, may be curved to conform to at least a portion of a coronal circumference or a portion of a sagittal circumference of the head. One or more therapy devices 370 can be adjustably attached to the cross element 330 to provide therapy to the patient for alleviating head pain. In an example, the therapy device 370 can be positioned at or next to a scalp site corresponding to a motor cortex target of the head, and to deliver tDCS to the motor cortex region.

Although only one cross element 330 is shown in FIG. 2, it is within the contemplation of the present inventors to include additional cross elements removably attached to the side elements 314A-B via respective connectors at different attachment positions. Therapy devices, such as 280A-D, can be detachably or adjustably attached to the additional cross elements, and configured to deliver various therapy modalities including, for example, thermal energy, ultrasonic energy, vibration or tactile massage, or high-frequency RF energy, among others. With the detachable and interchangeable cross elements, the therapeutic eyewear 300 can provide flexible and customized therapy combinations to meet the needs of different patients.

The rear element 316 can be made of elastic material, such as a flexible strap, to allow the base 310 to adjustably and securely fit to different head sizes. In an example, the rear element 316 may include attachment points to allow one or more therapy devices 360 to be detachably attached to the rear element. Examples of the therapy devices 360 can include transducers configured to deliver TENS to an occipital site innervated by an occipital nerve.

The therapeutic eyewear 300 may include one or more control compartments 340A-B, such as located on the base 310, along the side elements 314A-B, or on the rear element 316. Similar to the control compartment 240A-B as illustrated in FIG. 2, the control compartments 340A-B can include electrical and mechanical coupling interface and control circuitry that controls operation of various therapy devices, such as the therapy devices 350, 360 and 370. The control compartments 340A-B can additionally include transducers that provide therapeutic energies for alleviating head pain, or physiological sensors for sensing physiological signals from the head. Examples of the physiological sensors for use in the headgear are discussed below, such as with reference to FIG. 8. The control compartments 340A-B can also include an interface and/or a communication module for connecting or communicating with an external device or for charging the one or more batteries in the therapeutic eyewear 300.

FIGS. 4A-B illustrate, by way of example and not limitation, an example of a therapeutic headgear 400 and a part of the environment in which the therapeutic headgear 400 can be used. In particular, FIG. 4A illustrates the front view, and FIG. 4B illustrates the back view, of the therapeutic headgear 400 when it fits to a patient's head. The therapeutic headgear 400, which is an embodiment of the headgear 110 in FIG. 1, may include a number of detachable frame elements 430A-E each curved to conform to at least a portion of a circumference of the head. The frame elements 430A-E may each include one or more therapy devices removably or adjustably attached to the respective frame element. The frame elements 430A-E may be made of elastic material to allow the attached therapy devices to be in close contact with the target sites on the scalp or skin, while the therapy devices are securely held onto the respective frame element.

Similar to the therapy devices as previously discussed with reference to FIGS. 2 and 3, the therapy devices attached to the frame elements 430A-E can include a neuromodulator configured to deliver electrostimulation (such as TENS or tDCS) or magnetic stimulation (such as TMS), a thermal energy transducer configured to generate thermal energy, an ultrasound transducer configured to generate ultrasonic energy, a vibrotactile transducer configured to generate vibration or tactile massage, a RF transducer configured to generate high-frequency RF energy, a laser device configured to generate laser light energy, a visual stimulator configured to generate or prevent visual stimulation, or an aural stimulator configured to generate aural stimulation. In an example, the frame element 430A may be positioned at or next to the supra-orbital region of the patient's forehead, and include an attached neuromodulator configured to deliver TENS to a supra-orbital site innervated by a trigeminal nerve. In an example, the frame element 430D may be positioned at the occipital region of the head, and include an attached neuromodulator configured to deliver TENS to an occipital site innervated by an occipital nerve. In an example, the frame element 430C may be curved to conform to at least a portion of the coronal circumference across the motor cortex region of the head. The frame element 430C may include an attached neuromodulator configured to deliver tDCS to the motor cortex region. In an example, the frame elements 430B or 430D can each include thermal transducers to provide thermal therapy (cold or warm), ultrasound transducers to provide ultrasound energy, RF transducers to provide RF energy, a laser device to provide laser light energy, or vibrational transducers to provide vibrotactile energy, at the target sites on the scalp or skin.

The therapeutic headgear 400 may include two earpieces 440A-B that hold the detachable frame elements 430A-E together. The detachable frame elements 430A-E can be coupled to the 440A-B via connectors such as the connectors 113 as described with reference to FIG. 1. Similar to the control compartments 240A-B shown in FIG. 2, or the control compartments 340A-B shown in FIG. 3, the earpieces 440A-B can include one or more of an interface and control circuitry that controls various therapy devices, a communication module for connecting or communicating with an external device, one or more physiological sensors for sensing physiological signals, or transducers that can provide therapeutic energies for alleviating head pain. By way of non-limiting example, and as illustrated in FIG. 4, the two earpieces 440A-B can be sized and shaped to cover pinnae of the patient, and include an audio speaker configured to generate aural stimulation or auditory sound to facilitate the alleviation of head pain.

FIGS. 5A-B illustrate, by way of example and not limitation, an example of an electrode assembly 500 for delivering neuromodulation therapy for alleviating head pain. In particular, FIG. 5A shows the plan view, and FIG. 5B shows a cross sectional view of the electrode assembly 500. The electrode assembly 500 can be coupled to a therapy device configured for generating electrostimulation such as TENS or tDCS, such as the therapy devices 115A-B as shown in FIG. 1, the therapy devices 250, 260 or 270 shown in FIG. 2, the therapy devices 350, 360 or 370 shown in FIG. 3, or the therapy devices attached to one or more of the frame elements 430A, 430C, or 430D for delivering neuromodulation therapy at the target sites on the head as shown in FIG. 4.

The electrode assembly 500 can include a coupling element 510, an electrode body 520, and one or more electrode pins 530. The coupling element 510 can be shaped to enable a detachable engagement onto the base, or the extension such as the frame elements 230, the cross element 330 or the rear element 316, or the interchangeable frame elements 430A, 430C or 430D, of the headgear. Examples of the engagement can include a snap-fit coupling, a threaded or other rotation or screw-in coupling, a slide-in engagement, a hinge, or other adjustable coupling mechanisms.

The electrode body 520 can include a reservoir 540 that can be filled with conductive gel, saline, or other fluid, such as via a sealable opening 512 on the coupling element 510. The conductive gel or the saline can be used to promote electrical conductivity with the skin or scalp. Each of the electrode pins 530 may have a polymer outer coating, and an electrode tip 532 at a distal end of the electrode pin. The electrode tip 532 can be made of metal, or conductive polymer that provides a soft contact with the scalp or skin for enhanced patient comfort. As illustrated in FIG. 5B, at least one of the electrode pins 530 may have a lumen that forms a conduit 550 connecting the reservoir 540 and an opening on the electrode tip 532. The conduit 550 can conduct the conductive gel or saline to the electrode-tissue interface via the opening on the electrode tip 532. In an example, conductive gel or saline can be pre-filled into the reservoir 540 prior to placing the headgear on the patient's head. Once the headgear is worn on the head and the electrode assembly 500 properly positioned at the target site, the reservoir 540 can be squeezed, pushing the conductive gel or saline through the conduit 550 and spreading over the electrode-tissue interface via opening on the electrode tip 532. In another embodiment, the reservoir 540 may serve as a passive pump to slowly push out the conductive gel or saline from the electrode tip over a period of time, such as approximately 20-30 minutes.

In an example, the electrode pins 530 can include one or more spring-loaded electrode pins that may provide pressurized contact with the scalp or skin. The spring-loaded electrode can move independently and comply with varying contours of the head, thereby improving the electrical contact with the scalp through hair. In some examples, the spring-loaded electrode pins can have respective conduits for conducting the saline or conductive gel. When the spring-loaded electrode pins are in contact with the skin or scalp, the springs are pressed against the reservoir, causing a small amount of conductive gel or saline to be squeezed and released to the electrode-tissue interface. The use of conductive gel or saline and the spring-loaded electrode pins, either individually or in combination, may improve the electrical conductivity between the electrode and the scalp or skin, as well as improve patient comfort.

The electrode pins 530 can be electrically coupled to the control circuitry of the transducers and the power supplies, such as through wires extended through the electrode body 520. The electrode pins 530 can be configured to provide unipolar or bipolar electrostimulation. In an example, each electrode pin can be independently controlled to deliver electrostimulation at their respective locations. In another example, at least some electrodes can be electrically connected to each other to form a common polarity, such as an anode or cathode. For example, the electrode pins on the outer ring as shown in FIG. 5A can be electrically connected together to form one polarity (e.g., anode electrode), and a center electrode pin(s) surrounded by the outer electrode pins can form a different polarity (e.g., cathode). Bipolar electrostimulation can be delivered between the center electrode pin(s) and the outer electrode pins.

FIGS. 6A-B illustrate, by way of example and not limitation, an example of another type of electrode assembly 600A or 600B for delivering electrostimulation for treating head pain. The electrode assemblies 600A-B each has a shape of a comb that comprises an electrode body 620 and a plurality of teeth 630 extending outward from the electrode body 620. The teeth 630 may grip and retain the hair. The electrode assembly 600A or 600B each may have a coupling element 610 for detachable engagement onto the base or the extension of the headgear. At least some of the teeth 630 can have one or more electrode contacts disposed at the distal tip, or along the length, of the respective tooth. The teeth 630 can be made of elastic or superelastic material, such as Nitinol, for gentle gripping of hair on the scalp and allowing the electrode contacts to slip through or under the hair to make electrical contact with the scalp.

By way of non-limiting examples, FIG. 6A shows the comb-shaped electrode assembly 600A which includes the teeth 630 each having a tip electrode 632. In an example, the tip electrodes of a first subset of the teeth 630A can be electrically connected to each other to form a first common polarity (e.g., an anode or a cathode), and the tip electrodes of a second subset of the teeth 630B can be electrically connected together to form a second common polarity (e.g., a cathode or an anode) different from the first common electrode. The first and second electrically connected tip electrode subsets can be used to provide bipolar electrostimulation at the target site on the head. By way of non-limiting examples, FIG. 6B shows the comb-shaped electrode assembly 600B which includes the teeth 630 each having a tip electrode 632 and a proximal electrode 634 electrically disconnected from the tip electrode 632. The teeth 630 may each provide bipolar electrostimulation using the electrodes 632 and 634. In an example, the tip electrodes 632 of some of all of the teeth 630 can be electrically connected together to form a first common polarity, and the proximal electrodes 634 of some of all of the teeth 630 may be electrically connected together to form a second common polarity different from the first common polarity. The first and second electrically tied electrodes thus formed may be used to provide bipolar electrostimulation at the target site on the head. In an example, at least some of the teeth 630 can include respective reservoirs for storing conductive gel or saline, or spring-loaded electrodes, such as discussed with reference to the electrode assembly 500 in FIGS. 5A-B.

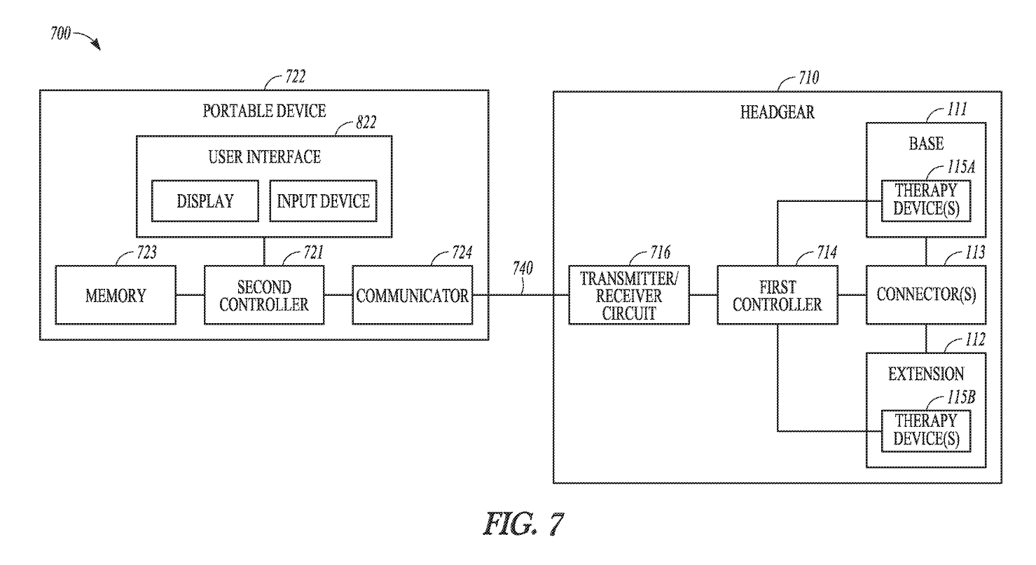

FIG. 7 illustrates, by way of example and not limitation, an example of a system 700 for non-invasive management of head pain. The system 700 can include a headgear 710, and a portable device 720 communicatively coupled to the headgear 710 via a communication link 740.

The headgear 710 can be sized and shaped to be worn on a patient's head for treating or alleviating head pain, and can be an embodiment of the headgear 110 as illustrated in FIG. 1, or the therapeutic cap 200 in FIG. 2, the therapeutic eyewear 300 in FIG. 3, the therapeutic headgear 400 in FIG. 4, or any variant thereof. Similar to the headgear 110, the headgear 710 may include the base 111, and the extension 112 coupled to the base 111 via the one or more connectors 113. The headgear 710 may include a first controller 714 which may be an embodiment of the first controller 114 of the headgear 110 and configured to control the two or more therapy devices 115A-B for delivering the respective modes of therapeutic energy to the respective target sites. The headgear 710 may additionally include a transmitter/receiver circuit 716 for establishing a communication with the portable device 720, such as receiving programming instructions from, or transmitting the sensory or therapeutic information to, the portable device 720.

The portable device 720 may include a user interface 722, a memory 723, a communicator 724, and a second controller 721 coupling to the user interface 722, the memory 723 and the communicator 724. Examples of the portable device 720 can include a personal mobile communication device, such as a mobile phone, a tablet, a laptop computer, or a handheld PDA, among other handheld or otherwise portable electronic devices. The second controller 721 may include a programmer circuit that can generate one or more therapy parameters for use by the therapy devices 115A-B on the headgear 710 in delivering various modes of therapy, such as electrostimulation, magnetic stimulation, thermal energy, ultrasound energy, RF energy, laser light energy, or vibrotactile energy, among others. In addition to programming individual therapy devices, the programmer circuit may additionally generate a therapy plan including timing and sequence of two or more therapy modalities. For example, the therapy plan may include delivering TENS and tDCS in an alternating mode, such as delivering TENS stimuli for a first duration (e.g., 10 seconds) followed by tDCS for a second duration (e.g., 10 seconds).

The user interface 722 can include a display and an input device. The display can produce a human-perceptible presentation of information including status of the therapy devices, such as their on/off states, positions on the head, existing parameter settings, etc. The presentation may also include programming options for the various therapy devices, such as therapy strength, dosage, time, or duration, among others. The information can be presented in a table, a chart, a diagram, or any other types of textual, tabular, or graphical presentation format. The presentation can include audio or other media format to alert the system user (e.g., the patient, or a clinician) of a therapy being delivered or withheld.

The input device of the user interface 722 enables the system user to create, select, deselect, confirm, override, or otherwise edit one or more therapy parameters, and save the personal therapy information. The input device of the user interface 722 may also enable the system user to adjust the presentation on the display. Examples of the input device can include keyboard, on-screen keyboard, touch-screen, mouse, trackball, touchpad, or other pointing or navigating devices. The programmer circuit of the second controller 721 can produce control signals according to the user input, and configure the communicator 724 to transmit the programming options to the therapy devices 115A-B. The second controller 721 may alternatively or additionally configure the memory 723 to store the user input in the memory 723. Examples of the user input stored in the memory 723 may include patient diaries on his/her head pain episodes, head pain trigger-avoidance tips, or patient personal therapy information including a plurality of therapy plans. Each therapy plan may include a pre-defined set of one or more therapy devices with their respective configurations and therapy parameters, and a schedule of delivery of the therapies (e.g., timing and sequence). The second controller 721 may configure the user interface 722 to present the stored therapy plans on the display, and to receive user selection or modification of one or more therapy plans via the input device. The second controller 721 can generate the therapy control signals in accordance with the user selected therapy plan and configure the communicator 724 to transmit the therapy control signals to the corresponding therapy devices on the headgear 710 to deliver respective therapy to the patient.

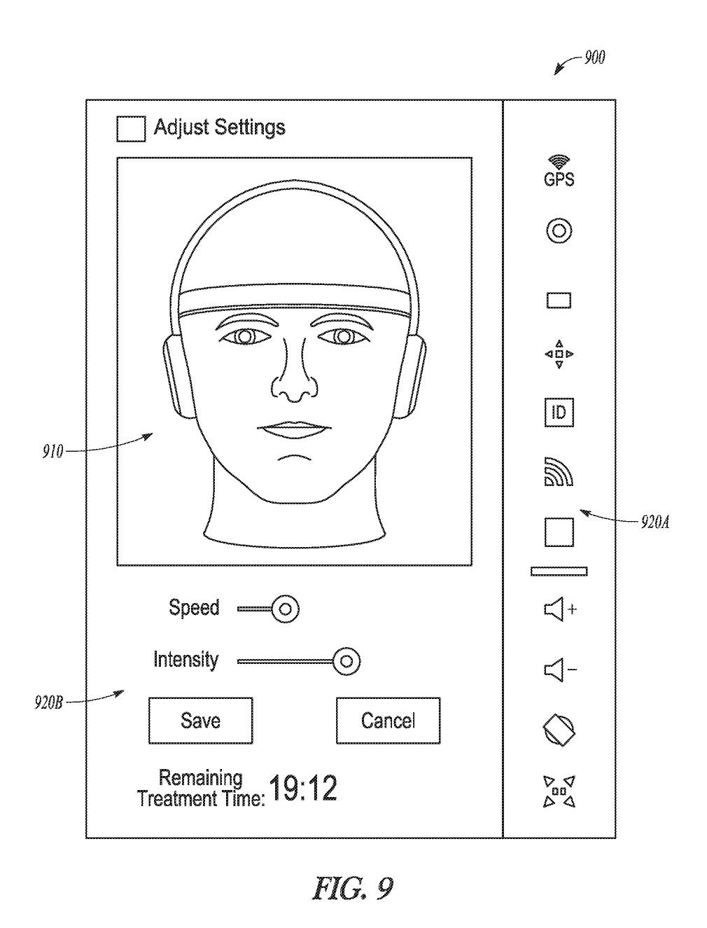

The communicator 724 can be configured to communicate with at least the therapy devices 115A-B on the headgear 710 through the communication link 740. The communication link 740 can be a wired connection, such as a universal serial bus (USB) connection, or otherwise cables coupled to a communication interface on the communicator 724. The communication link 740 can be a wireless connection such as Bluetooth protocol, IEEE 802.11 wireless, an inductive telemetry link, or a radio-frequency telemetry link, among others. Examples of the portable device for communicating with the headgear are discussed below, such as with reference to FIG. 9.

FIG. 8 illustrates, by way of example and not limitation, an example of a system 800 for non-invasive management of head pain. Similar to the system 700, the system 800 can include a headgear 710 and a portable device 820 communicatively coupled to the headgear 710 via the communication link 740. The system 800 can additionally include a web-based data repository 830, and/or one or more physiologic sensors 817. The web-based repository 830 can be stored and maintained in a server accessible via the Internet. The web-based repository 830 can store public therapy information obtained from two or more users. The public therapy information can be organized and presented in one or more formats such as a head pain discussion group, a headgear users' forum, a therapy device programming information exchange platform, or other cyber communities established based on various head pain conditions or treatment.

The portable device 820, which is an embodiment of the portable device 720 as illustrated in FIG. 7, can additionally include an Internet access module 825 to establish a wired or wireless connection to the Internet 850. In an example, the portable device 820 is a personal mobile communication device that can wirelessly connect to the Internet using an IEEE 802.11 wireless network, cellular network, satellite network, or microwave network. In an example, the connection to the web-based data repository 830 can be through a secured Internet connection. The second controller circuit 821 can execute software programs such as a web browser or an application (“App”) on a mobile communication device. Upon receiving a user input via the user interface 822 (such as by inputting the web address of the data repository 830), the second controller circuit 821 can configure the Internet access module 825 to establish the Internet connection 850 to the web-based data repository 830, and enable the patient to access content in the web-based data repository 830, which can be displayed on the user interface 822. The Internet access module can include hardware, such as controlled by software, for accessing the Internet including, for example, dial-up with a modem via telephone circuits, broadband over coaxial cable, fiber optic or copper wires, Wi-Fi, or satellite or cellular telephone technology, among others. The second controller circuit 821 can additionally or alternatively configure the Internet access module 825, upon receiving respective user inputs, to upload the patient's personal therapy information onto the data repository 830, or to download from the data repository 830 the public therapy information into the portable device 820. In an example, the data repository 830 may contain information about other patients' head pain symptoms. The patient may identify from the data repository 830 those patients whose symptoms are similar to that of the patient's, review the settings of their headgear (such as the positions and therapy parameters associated with the therapy devices), therapy outcome, and other patients' evaluations. The data repository 830 may contain rankings of various therapy plans (activation or deactivation of therapy devices, corresponding therapy parameters, and scheduling of the therapy delivery). The patient may make various queries, and choose to download a therapy plan into the portable device 820. The downloaded information can be stored in the memory 823, or be used to program the therapy devices on the headgear 710 via the communication link 740.

The one or more physiologic sensors 817 can be configured to sense one or more respective physiological signals from the patient. Examples of the physiological signals can include a heart rate signal, a pulse rate signal, a heart rate variability signal, a galvanic skin response (GSR) signal, a skin temperature signal, an electromyogram (EMG) signal, an electroencephalogram (EEG) signal, a magnetoencephelogram (MEG) signal, a hemodynamic signal such as a blood flow signal, a blood pressure signal, a blood perfusion signal, or a photoplethysmography (PPG) signal, or a saliva production signal indicating the change of amount of saliva production, among others. In an example, one or more of the physiologic sensors 817 can be detachably or adjustably attached to the headgear 710, such as on the base 111 or the extension 112. In an example, one or more of the physiologic sensors 817 can be implantable, wearable, or otherwise separable from the headgear 710. The physiological sensors 817 can be communicatively coupled to the portable device 820, such as through the communication link 740, or through a separate communication pathway and communication interface different from the communication link 740.

The portable device 820 can includes a signal processor 826 configured to detect from the one or more physiological signals, such as received from the physiological sensors 817, a physiological event indicative or predictive of an onset of a head pain episode, or worsening or improvement of an existing head pain episode. The physiological signals or the detected physiological events can be displayed on the user interface 822. In response to the detected physiological event, the second control circuit 821 can generate an alert, such as an audio, visual, or other formats of message on the user interface 822, to warn the patient of an impending head pain episode, to recommend the patient take preventive actions, seek medical care, or wear the headgear 710 and activate a preventive therapy session using one or more therapy devices such as the therapy devices 215A-B. Such preventive therapy can disrupt the patient's usual progression or sequence of aberrant physiological processes such as to preemptively abort the onset of a head pain episode. In an example, the preventive therapy session can include TENS or tDCS with specified parameter values. For example, elevated levels of the neuropeptide calcitonin gene-related peptide (CGRP) may be associated with the pathophysiology of migraine attacks. A physiological sensor can be used to detect an aberrant CGRP level such as through a saliva test, and alert the patient of the aberrant CGRP level, or to recommend the patient to initiate tDCS sessions to reduce the CGRP levels, such that future head pain episodes may become less frequent. By way of example and not limitation, the tDCS therapy in this example is prophylactic, instead of abortive, in nature. That is, the patient is not experiencing a migraine episode during the tDCS therapy session as he or she would if the therapy were being used to abort an in-progress head pain episode.

In an example, the signal processor 826 can detect the physiological event during an on-going therapy session when the headgear 710 is used by the user. The detected physiological event may indicate a side effect of therapy (such as pain or discomfort caused by inadequate or excessive stimulation strength, or stimulation of non-target tissue), or lack of desired therapy efficacy. Based on the detected physiological event, the second control circuit 821 may generate an alert on the user interface 822 prompting the patient to adjust the spatial positions of the therapy devices (or the associated electrodes) on the headgear, or to adjust one or more therapies parameters either automatically or at least partially through user input. For example, in monitoring the cortical spreading depression from an EEG signal, a detection of a spike followed by a significant reduction in EEG may be predictive of an upcoming migraine attack. The second controller 821 may produce an alert, and a recommendation for immediate use and activation of the headgear 710. In an example, a detection of depressed heart rate variability may indicate elevated sympathetic tone and a state of patient stress, and increased risk of migraine attacks. In another example, galvanic skin response (GSR) can be indicative of skin conductivity. The GSR may also be referred to as electrodermal activity (EDA). An elevated GSR may be a result of sweating, indicating increased sympathetic tone or elevated state of patient stress. In another example, the EMG may be monitored to detect muscle capture. If muscle capture corresponding to pain is realized, the second controller 821 may adjust one or more therapy parameters to avoid or reduce muscle capture. Adjustment of therapy parameters or spatial positions of the therapy devices can also be based on other physiological measurements such as changed jaw pressure, or sleep patterns.

FIG. 9 illustrates, by way of example and not limitation, an example of a mobile communication device 900 for controlling a headgear for head pain management, such as the headgears 200, 300, 400, or 710, via a wired or wireless connection. The mobile communication device 900, such as a mobile phone, can be an embodiment of the portable device 720. The mobile communication device 900 may include a display 910 and a number of user input devices such as touch-screen controls 920A-B. The display controls 920A can enable the user to select or adjust the presentation on the display 910. The mobile communication device 900 can execute software, such as a mobile application (“App”), to enable the user to establish communication with the headgear, activate or deactivate one or more therapy devices on the headgear, and adjust therapy parameters for one or more therapy devices. The software or the mobile App may provide the user with the capability to modify, save, recall, or adaptively learn the most preferred therapy-parameter sets for each individual. In an example, the App can automatically learn over time by monitoring patient preferences during head pain therapy sessions (such as therapy device positioning within the headgear, therapy type, therapy parameters, etc.), and make suggestions for individualized parameter values that are tailored to the user. The App can store the information about the learned therapy sessions such as specific locations of the therapy devices, device types at respective location, or therapy parameters, among others. For example, the system would recognize that a tDCS therapy device was programmed and used at a specific location along the headgear. If a patient tries to recall and reuse that same therapy session, but had the tDCS therapy device in a different location or programmed with different parameters, the App may alert the patient of that situation. The mobile communication device 900 can optionally control the physiological sensors 817 for collecting physiological signals.

In an example, the mobile communication device 900 has an embedded camera allowing the user to take a picture of the user wearing the headgear, and display the picture on the display 910. The patient is enabled to touch the therapy device as shown in the picture, such as the neuromodulators or other transducers on the headgear, to select the corresponding actual therapy devices. Appropriate context-sensitive touch-screen therapy controls 920B corresponding to the selected therapy device may be displayed on the display, such as speed or frequency, intensity, or duration for TENS, tDCS, TMS, or other neuromodulation therapies, or heat intensity for thermal therapy, volume for aural stimulation, vibrational strength, frequency, or pattern, among others. The user can use the touch-screen therapy controls 920B to select or edit the therapy parameters to match his or her preference. The user can save the selected therapy devices settings and associated therapy parameters as a therapy plan in the memory, or retrieve from memory one or more previously stored therapy plans. The mobile communication device 900 can generate control signals in accordance with the user's selection and programming of the therapy devices to control the therapy devices residing on the headgear via a wired connection such as a USB connection, or a wireless connection such as the Bluetooth protocol.

In some examples, similar to the portable device 820 in accessing the web-based data repository 830 with reference to FIG. 8, the mobile communication device 900 may include a module (which may include telecommunication hardware or software) for establishing an Internet connection to the web-based repository 830. The user is enabled to access the public therapy information stored in the web-based repository 830, participate and exchange information in the cyber communities of head pain patients and headgear users, upload the patient's personal therapy information onto the data repository 830, or to download from the data repository 830 the public therapy information into the mobile communication device 900.

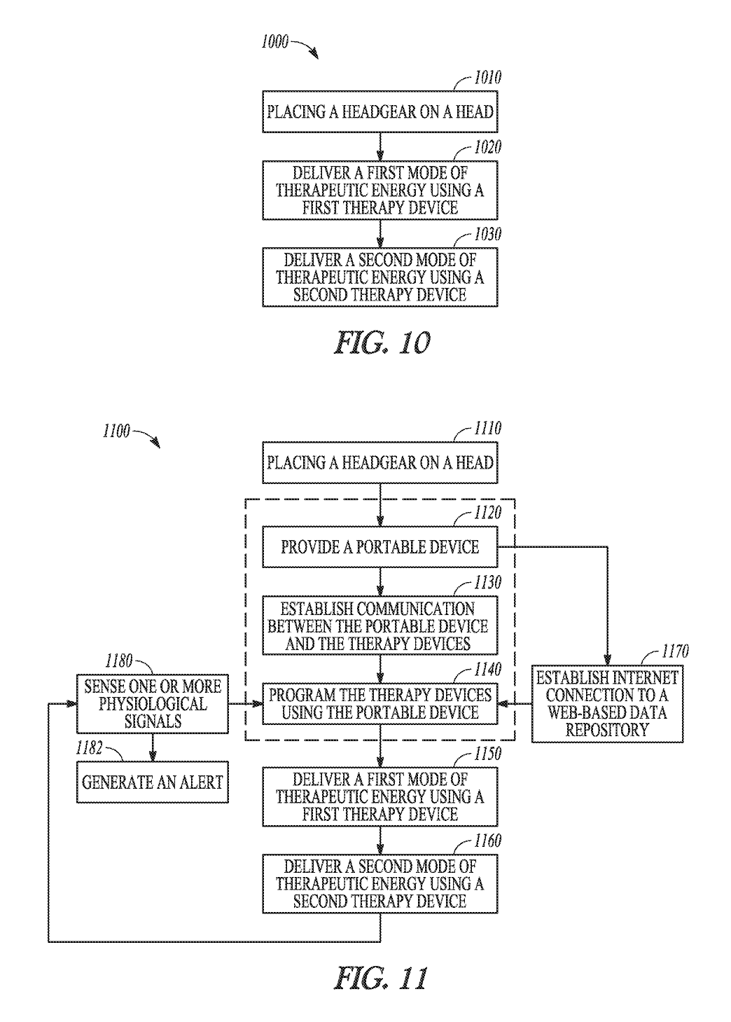

FIG. 10 illustrates, by way of example and not limitation, an example of a method 1000 for non-invasively managing head pain in a patient using a headgear wearable on a patient's head. The method 1000 may be used to operate the headgear 110 as illustrated in FIG. 1, or the therapeutic cap 200 in FIG. 2, the therapeutic eyewear 300 in FIG. 3, the therapeutic headgear 400 in FIG. 4, or any variant thereof.

The method 1000 begins at 1010, where a headgear may be placed on the head of the patient. As previously discussed, such as with reference to any one of FIGS. 2-4, the headgear may include structural members such as a base and one or more frame elements to which two or more therapy devices can be detachably or adjustably attached. The therapy devices can be configured to provide various modalities of therapies to treat or relieve symptoms of migraines, cluster headache, tension headache, or sinus headache, among other types of headaches. Examples of the therapy modalities can include electrostimulation or magnetic stimulation therapy, or therapies that employ various types of energy such as magnetic energy, thermal energy, radio-frequency energy, ultrasonic energy, vibrotactile energy, or laser light energy, among others. The headgear can be worn on the patient's head when the patient is indicated to suffer an on-going head pain episode, or to experience an impending head pain episode such as predicted by one or more physiological sensors or provided by a healthcare professional. The headgear and its associated therapy devices can also be utilized in a prophylactic mode, such as to prevent or decrease the frequency and intensity of future occurrences of head pain.

The therapy devices attached to the headgear may be positioned to their respective desired target locations on the scalp or the skin. In some examples, additional therapy devices can be attached to the headgear, or some attached therapy devices can be removed from the headgear. The therapy devices can be moved around and repositioned relative to the base or the frame elements of the headgear. This can provide enhanced flexibility in applying head pain therapies with desired number and location of the therapy sites. In an example, a first therapy device can be a neuromodulator configured to generate and deliver transcutaneous electrical nerve stimulation (TENS). The first therapy device, or an electrode coupled to the first therapy device, can be positioned at or next to the supra-orbital region of the patient's forehead, or at the occipital region of the head. In an example, a second therapy device can be a neuromodulator configured to generate and deliver transcranial direct current stimulation (tDCS). The second therapy device, or an electrode coupled to the second therapy device, can be positioned at the scalp site corresponding to a motor cortex target of the head. Other therapy devices, such as transducers configured to provide various modalities of therapies, can be positioned at respective target sides on the head, such as previously discussed with reference to FIGS. 2-4.

At 1020, a first mode of therapeutic energy can be delivered at the target site on the patient's head using a first therapy device. In an example, TENS stimuli can be delivered at the supra-orbital region of the patient's forehead to modulate a trigeminal nerve, or at the occipital region of the head to modulate an occipital nerve. TENS can disrupt the mechanism causing head pain by swamping the pain fibers with non-noxious peripheral stimuli, thereby shutting the pain gate at the junction of the peripheral and central nervous system, and blocking the pathway (such as via the trigeminal nerve and/or the occipital nerve) leading to the CNS where the pain sensation is interpreted. In an example, TENS stimuli can be programmed to have one or more time-variant stimulation parameters. The time-variant stimulation parameters can include time-variant duty cycle, time-variant pulse amplitude, time-variant pulse width, time-variant frequency, or time-variant stimulation duration, among others. In an example, the time-variant stimulation parameters can include a random stimulation parameter such as random burst duty cycles. The time-variant or random stimulation parameters may reduce the likelihood of complications such as neural accommodation, adaptation, habituation, or stimulus generalization, which may cause diminished neural response to stimulation and therapeutic efficacy over time.

At 1030, a second mode of therapeutic energy can be delivered at the target site on the patient's head using a second therapy device. In an example, tDCS stimuli can be delivered at the scalp site corresponding to a motor cortex target of the head. The tDCS via motor cortex may impact indirectly, through interaction with interneurons, deep-brain structures associated with head-pain transmittal, thereby reversing central sensitization of pain. TENS and tDCS therapy may be delivered in an alternating mode. For example, TENS can be delivered for a first duration (e.g., 10 seconds) followed by tDCS for a second duration (e.g., 10 seconds). Other therapy devices, such as transducers to provide thermal energy, ultrasound energy, RF energy, laser light energy, vibrotactile energy, or visual or aural stimulation, may also be used. Various modalities of head pain therapies can be delivered according to a therapy plan that defines activation or deactivation of the therapy devices, their respective configurations and therapy parameters, and a schedule of delivery of the therapies (e.g., timing and sequence).

FIG. 11 illustrates, by way of example and not limitation, an example of a method 1100 for non-invasively managing head pain in a patient using a system comprising a headgear and a portable device. The method 1100 may be implemented in, or be used to operate the head pain management systems 700 or 800, as respectively illustrated in FIGS. 7-8.

Similar to the method 1000, the method 1100 begins at 1110 with placing a headgear on the head of the patient, where two or more therapy devices attached to the headgear may be positioned to their respective desired target locations on the scalp or the skin. At 1120, a portable device can be provided, which can be a mobile phone, a tablet, a laptop computer, or a handheld PDA, among other handheld or otherwise portable electronic devices. The headgear can have a transmitter/receiver circuit, such as the transmitter/receiver circuit 716 as illustrated in FIGS. 7-8, for establishing a communication with the portable device via a communicator, such as the communicator 724 or 824 as illustrated in FIGS. 7-8. At 1130, a communication between the portable device and one or more therapy devices on the headgear can be established. The communication can be through a wired communication connection such as a cable coupled to a communication interface on the communicator, or a wireless connection such as the Bluetooth protocol, IEEE 802.11 wireless, an inductive telemetry link, or a radio-frequency telemetry link, among others. In an example, the portable device is a mobile phone, such as illustrated in FIG. 9, which has a USB connection or a Bluetooth wireless connection to the therapy devices on the headgear.

At 1140, one or more therapy devices on the headgear can be programmed using the portable device. The portable device can execute pre-installed software, such as a mobile application (“App”), to enable the user to select, activate, or deactivate one or more therapy devices on the headgear, and adjust therapy parameters for one or more therapy devices. Examples of the therapy parameters for neuromodulators delivering TENS may include pulse amplitude, pulse width, frequency, duty cycle, or stimulation duration, among others. Examples of the therapy parameters for other transducers may include current intensity for tDCS heat intensity for thermal therapy, volume for aural stimulation, vibrational strength, frequency, or pattern for vibrotactile therapy, or duration of therapy, among others. The user may also program a therapy plan including scheduling timing or sequence of various therapy modalities. Additionally or alternatively, the user is enabled to retrieve from the memory of the portable device stored configurations or parameter values for the therapy devices or stored therapy plans, edit and save the therapy parameters or therapy plans, and program the therapy devices. The patient may also create and save to the memory of the portable device diaries on his/her head pain episodes, head pain trigger-avoidance tips, or patient personal therapy information including a plurality of therapy plans.

The method 1100 may include a step for detecting one or more physiological signals, such as by using one or more implantable, wearable, or other ambulatory physiological sensors. The physiological sensors may be detachably or adjustably attached to the headgear, or separate from the headgear. At 1180, one or more physiological signals can be sensed by physiological sensors, including a heart rate signal, a pulse rate signal, a heart rate variability signal, a galvanic skin response signal, a skin temperature signal, an electromyogram (EMG) signal, an electroencephalogram (EEG) signal, a magnetoencephelogram (MEG) signal, a hemodynamic signal, or a saliva production signal indicating the change of amount of saliva production, among others. The sensed physiological signals can be processed such as by a signal processor in the portable device to detect a physiological event indicative of or predictive of an onset of a head pain episode. For example, in monitoring the cortical spreading depression from an EEG signal, a detection of a spike followed by a significant reduction in EEG may be predictive of an impending migraine attack. In an example, a detection of depressed heart rate variability may indicate elevated sympathetic tone and a state of patient stress, and increased risk of migraine attacks. In another example, an elevated galvanic skin response (GSR) may be a result of sweating, indicating increased sympathetic tone or elevated state of patient stress. In another example, a combination of two or more physiological parameters may produce improved capability of detecting an impending head pain attack. An alert of an impending head pain attack can be generated at 1182. The alert can be an audio, visual, or other format of message presented to the user, recommending the patient to put on and activate the headgear (if the headgear is not being worn or activated). The detection of the physiological event and the alert can then be used to program the therapy devices at 1140.

At 1150, a first mode of therapeutic energy using a first therapy device, such as TENS stimuli can be delivered at the supra-orbital region of the patient's forehead to modulate a trigeminal nerve, or at the occipital region of the head to modulate an occipital nerve. At 1160, a second mode of therapeutic energy can be delivered using a second therapy device, such as tDCS stimuli delivered at the scalp site corresponding to a motor cortex target of the head. Additional modalities of therapies can also be delivered according to the therapy parameters or the therapy plans.

During the delivery of the first, second, or other modalities of head pain therapies, one or more physiological signals can be sensed at 1180. Unlike the detection of a physiologic event indicative of or predictive of an impending head pain episode, the detected physiological event during a therapy session of an on-going head pain episode may indicate a side effect of therapy (such as pain or discomfort caused by inadequate or excessive stimulation strength, or stimulation of non-target tissue), progression of the existing head pain episode, or lack of desired therapy efficacy. For example, an EMG may be monitored to detect muscle capture during electrostimulation therapy. If muscle capture corresponding to pain is realized, the therapy parameters can be adjusted at 1140 to avoid or reduce muscle capture. An alert can be generated at 1182, prompting the patient to adjust the spatial positions of the therapy devices (or the associated electrodes) on the headgear, or re-program one or more therapy devices, such as modifying stimulation parameters associated with one or more therapy devices. Other physiological events indicative of changed jaw pressure or sleep patterns can also be detected to provide feedback to adaptively adjust therapy parameters or reposition the therapy devices on the head.

The method 1110 can optionally include a step 1170 for establishing an Internet connection to a web-based data repository, where public therapy information obtained from two or more users can be stored. The public therapy information can be organized in one or more formats of a head pain discussion group, a headgear users' forum, a therapy device programming information exchange platform, or other cyber communities. The user is enabled to access the web-based repository, participate and exchange information in the cyber communities of head pain patients and headgear users, upload the patient's personal therapy information onto the data repository, or to download from the data repository the public therapy information into the portable device. The downloaded information can be stored in the memory of the portable device, or be used in programming the therapy devices at 1140.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Some processes described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A system for non-invasive management of head pain, comprising:
a headgear configured to be worn on a patient's head, the headgear including: a base;
an extension coupled to the base via one or more connectors;
at least first and second therapy devices removably or adjustably attached to one or more of the base or the extension, the first therapy device configured to deliver a first mode of therapeutic energy to a first target site on the head, the second therapy device configured to deliver a second mode of therapeutic energy to a second target site on the head; and
a first controller attached to the base, the first controller configured to control the at least first and second therapy devices for delivering the respective first and second modes of therapeutic energy to the respective first and second target sites.

2. The system of claim 1, wherein the extension includes one or more frame elements detachably or adjustably connected to the base via the one or more connectors, and wherein one or more of the at least first and second therapy devices are removably or adjustably attached to the one or more frame elements.

3. The system of claim 2, wherein:
the first therapy device is removably or adjustably attached to the base curved to conform to at least a portion of a transverse circumference of the head;
the second therapy device is removably or adjustably attached to one of the one or more frame elements of the extension, the one frame element curved to conform to at least a portion of a coronal circumference or a portion of a sagittal circumference of the head.

4. The system of claim 2, wherein the extension includes a cover securely interconnecting the base and the one or more frame elements of the extension.

5. The system of claim 2, wherein:
the base includes:
an eyepiece positioned close to one or both eyes of the patient;
at least two side elements connected to opposite sides of the eyepiece; and
a rear element connected to the two sidebars, and
the one or more frame elements include at least a cross element removably or adjustably attached to the two sidebars of the first frame element.

6. The system of claim 1, wherein the first and second therapy devices are respectively selected from the group consisting of:
a first neuromodulator configured to deliver transcutaneous electrical nerve stimulation (TENS);
a second neuromodulator configured to deliver transcranial direct current stimulation (tDCS);
a third neuromodulator configured to deliver transcranial magnetic stimulation (TMS);
a thermal energy transducer configured to generate thermal energy;
an ultrasound transducer configured to generate ultrasonic energy;
a vibrotactile transducer configured to generate vibration or tactile massage;
a radio-frequency (RF) transducer configured to generate high-frequency RF energy,
a laser device configured to generate laser light energy;
a visual stimulator configured to generate or prevent visual stimulation; and
an aural stimulator configured to generate aural stimulation.

7. The system of claim 6, wherein:
the first therapy device includes the first neuromodulator configured to deliver TENS to a first target site innervated by a trigeminal nerve or an occipital nerve; and
the second therapy device includes the second neuromodulator configured to deliver tDCS to a second target site corresponding to a motor cortex target of the head.

8. The system of claim 6, wherein the TENS includes a pulse train with one or more time-variant stimulation parameters.

9. The system of claim 1, further comprising a portable device, the portable device including:
a communicator configured to communicate with the at least first and second therapy devices on the headgear via a transmitter-receiver circuit of the headgear;
a user interface including a display and an input device configured to receive user input;
a second controller coupled to the user interface and the communicator, the second controller including a programmer circuit configured to generate one or more therapy parameters associated with the delivery of the first and second modes of therapeutic energy; and
a memory configured to store personal therapy information of the patient including the one or more therapy parameters.

10. The system of claim 9, wherein the portable device includes a personal mobile communication device communicatively coupled to the headgear through a wired or wireless connection.

11. The system of claim 9, further comprising:
one or more physiological sensors coupled to the portable device, and configured to sense respective one or more physiological signals;
the portable device includes a signal processor configured to detect from the one or more physiological signals a physiological event; and
the programmer circuit of the portable device is configured to initiate, or adjust the parameters associated with, the delivery of the respective first and second modes of therapeutic energy in response to the detected physiological event.

12. The system of claim 9, wherein the portal device is communicatively connected to a web-based data repository configured to store public therapy information obtained from two or more users, the portable device configured to enable the patient to:
access content in the web-based data repository;
upload personal therapy information of the patient onto the data repository; or
download from the data repository the public therapy information into the portable device for storing in the memory or programming the at least first and second therapy devices.

13. A method for non-invasively managing head pain in a patient using a headgear wearable on a patient's head, the method comprising:
placing the headgear on the patient's head;
positioning at least a first therapy device removably or adjustably attached the headgear to a first target site on the head;
positioning at least a second therapy device removably or adjustably attached the headgear to a second site on the head;
delivering a first mode of therapeutic energy at the first target site using the at least first therapy device; and
delivering a second mode of therapeutic energy at the second target site using the at least second therapy device.

14. The method of claim 13, further comprising:
providing a portable device configured to communicate with the at least first and second therapy devices on the headgear;
establishing a communication between the at least first and second therapy device and the portable device; and
programming the at least first and second therapy device using the portable device,
wherein delivering the first and second modes of therapeutic energy includes delivering the first and second modes of therapeutic energy using the respective first and second therapy devices according to the programming.

15. The method of claim 14, wherein the communication between the at least first and second therapy device and the portable device includes a wired or wireless connection.

16. The method of claim 13, wherein the at least first and second modes of therapeutic energy are respectively selected from the group consisting of:
a transcutaneous electrical nerve stimulation (TENS);
a transcranial direct current stimulation (tDCS);
a transcranial magnetic stimulation (TMS);
a thermal energy mode;
an ultrasonic energy mode;
a vibration or tactile massage mode;
a high-frequency RF energy mode;
a laser light energy mode;
a visual stimulation; and
an aural stimulation.

17. The method of claim 16, wherein:
delivering the first mode of therapeutic energy includes delivering the TENS to a first target site innervated by a trigeminal nerve or an occipital nerve; and
delivering the second mode of therapeutic energy includes delivering the tDCS to a second target site corresponding to a motor cortex target of the head.

18. The method of claim 17, wherein the TENS includes a pulse train with one or more time-variant stimulation parameters.

19. The method of claim 13, further comprising detecting a physiological event,
wherein delivering the first and second modes of therapeutic energy includes initiating, or adjusting one or more parameters associated with, the delivery of the respective first and second modes of therapeutic energy in response to the detection of the physiological event.

20. The method of claim 13, further comprising one or more of:
establishing a communication between the portable device and a web-based data repository that stores public therapy information obtained from two or more users;
using the portable device to access content in the web-based data repository;
uploading personal therapy information of the patient onto the data repository; or
downloading from the data repository the public therapy information into the portable device for storing in the portable device or programming the at least first and second therapy devices.
